(19) United States
(12) Patent Application Publication
(10) Pub. No.: US 2017/0143393 A1
(43) Pub. Date: May 25, 2017

(54) FIXING DEVICE FOR BONE FRACTURES

(71) Applicant: HIPP MEDICAL AG, Kolbingen (DE)

(72) Inventors: Markus HIPP, Kolbingen (DE); André WIRTH, Kolbingen (DE)

(21) Appl. No.: 15/309,849

(22) PCT Filed: May 21, 2015
(86) PCT No.: PCT/EP2015/061319

§ 371 Date: Nov. 9, 2016

(30) Foreign Application Priority Data
May 27, 2014 (DE) 10 2014 107 497.1

(51) Int. Cl. A61B 17/80

(52) U.S. Cl. CPC A61B 17/8009; A61B 17/8023; A61B 17/8047; A61B 17/8085; A61B 2017/681

(57) ABSTRACT

A fixing device for bone fractures having at least two tensioning elements, at least two fastening elements and at least two sleeve elements. Each of the tensioning elements has a contoured body and has, at ends mutually spaced apart in the longitudinal direction, receivers which enclose sleeve elements and/or fastening elements, so that by linking a plurality of tensioning elements, sleeve elements and/or fastening elements they can be arranged into a chain. Each tensioning element is formed with mirror-symmetry with respect to the longitudinal direction, wherein the receivers extend, starting from an imaginary center line of each tensioning element in longitudinal direction, upwards or downwards to the outer edge of the contoured body of the tensioning element in the height direction and have a toothing system on their contact surface facing the imaginary center line. When connecting individual tensioning elements, these can be aligned with respect to one another in a predetermined angular position by mutual engagement of the toothing system formed on mutually facing contact surfaces of the receivers.

The present invention relates to a fixing device for bone fractures according to claim 1.

It is known that, when a bone fracture is immobilized insufficiently, a callus formation, i.e. a tyloma-like thickening of the ends of the fracture from overgrowing bone tissue, can occur. In order to avoid such an indirect fracture healing via a callus, bone plates are used which are applied and attached to the outer surface of a broken hone so that the fracture site is fixed during the healing process.

For such applications for the treatment of bone fractures, from the prior art rigid, flat metallic bone plates having bores are mainly known, which are screwed at the opposite sides of a fracture. For the fastening of such fixations, so-called cortical screws are usually used. These screws are screwed into the outer bone tissue, the so-called cortical layer, which has the highest strength of the bone. The fixation ensures that the ends of the fracture cannot move with respect to each other and that newly formed bone tissue can accumulate without being subjected to loading.

Furthermore, it is assumed that the healing process of a fracture can be influenced beneficially when compression is applied onto the fracture site which has been fitted together. As a result, a particularly close adaptation, i.e. a very small clearance over which the ends of the fracture grow towards each other again, is effected.

A large number of different approaches for immobilizing a bone fracture can be found in the prior art.

The applicant who works intensively with a multiplicity of medical instruments has also achieved developments in the field of bone plates or a fixing device for bone fractures.

For instance, DE 10 2011 001 016 A1 by the applicant discloses a bone plate modular system in which a plurality of tensioning elements, which consist of a hollow contoured body, are connected to one another according to a type of tongue-and-groove connection such that they enclose a screw or the like which can be inserted through the contoured body. By combining a plurality of tensioning elements, they can be arranged into a chain or in the shape of a star or in any combination of chain and star arrangements. However, it is not possible to fix the relative alignment of the individual tensioning elements with respect to one another, whereby individual tensioning elements can be displaced, which is undesirable.

In order to overcome this problem, the applicant has proposed a different embodiment of a fixing device for bone fractures. This fixing device which is known from DE 10 2012 105 123 A1 likewise comprises a plurality of tensioning elements which consist of a hollow contoured body and whose ends which are mutually spaced apart in the longitudinal direction of the tensioning element have receivers formed thereon, into which fastening means can be inserted. In order to fix the individual tensioning elements, in dependence upon the type and complexity of the fracture to be treated, in a corresponding angular position with respect to one another, the fixing device disclosed in DE 10 2012 105 123 A1 proposes forming an internal toothing system in the receivers and, prior to fastening the tensioning elements to the bone by means of corresponding screws, connecting the mutually connected and correspondingly aligned tensioning elements by introducing a sleeve, which is provided with an outer toothing system, in an accurately fitting manner into the receivers. By combining the sleeves and tensioning elements with one another, the angular position is fixed and the fixing device assembled in this manner can be fastened to the ends of the fracture by means of corresponding screws.

A further embodiment of bone plates or fixing devices for bone fractures by the applicant is known from DE 10 2011 001 018 A1 which discloses a tensioning element for fixing devices which consists substantially of an continuous loop which is flexible and substantially not plastically deformable and through which at least two cortical screws can be inserted, and which has a stretch element having a variable length which can be inserted into the continuous loop and can be splayed within the continuous loop such that in order to apply forces, which are directed towards one another, to the fastening means by means of the stretch element a predetermined tensile stress can be produced over the circumference of the continuous loop.

Finally, DE 10 2012 105 125 A1 by the applicant discloses a bone plate comprising joints which permit a relative position change of the individual sections of the bone plate with respect to one another.

In particular, by reason of the varied requirements of this particularly sensitive application, such bone plates or fixing devices which are known from the prior art have a complex structure consisting of a multiplicity of different components and therefore the production of such “systems” is correspondingly complex and cost-intensive.

Therefore, in medical care only a limited group of patients or types of fractures have hitherto benefited from the advantageous application of a compression-producing fixation.

Therefore, the object of the invention is to provide a fixing device for bone fractures which can be produced in a particularly cost-effective manner, in particular in mass production, and is suitable for extending the range of application of compression-producing fixations of bone fractures.

The object is achieved by the fixing device for bone fractures according to claim 1. Advantageous embodiments are described in the dependent claims.

In accordance with the invention, a fixing device for bone fractures is proposed, comprising: at least two tensioning elements, at least two fastening means which, preferably in the region of ends of the fracture, can be introduced into the bone, and at last two sleeve elements, wherein each tensioning element has: a contoured body which is hollow in plan view, has a circumferential wall and has two lateral flanks which lie opposite one another in the width direction of the tensioning element and which, as seen in plan view, are directed in an arcuate manner outwards from an imaginary centre point of the tensioning element, wherein the lateral flanks merge seamlessly into Y-shaped connection points which run out into integrally formed receiving sections which, as seen in plan view, are directed in an arcuate manner inwards to the imaginary centre point of the tensioning element, at least two mutually spaced apart annular receivers which lie opposite one another preferably in the longitudinal direction of the tensioning element and which serve to receive the sleeve elements and/or the fastening means which can each be inserted through the receivers, wherein the receiving sections are an integral component of the annular receivers, so that they are formed in one piece with the contoured body and enclose a sleeve element and/or fastening means, and wherein by linking a plurality of tensioning elements, sleeve elements and/or fastening means, these can be arranged into a chain; wherein the circumferential wall of the contoured body of each tensioning element is deformable in a spring-elastic manner at least in sections in the region of the lateral flanks, wherein each tensioning element is formed with point-symmetry in the width direction or with mirror-symmetry with respect to the longitudinal direction of the tensioning element and the mutually spaced part receivers which lie opposite one another preferably in the longitudinal direction of the tensioning element are configured in such a manner that a receiver, preferably starting from an imaginary centre line of each tensioning element in the longitudinal direction, extends in the direction of the outer edge of the contoured body of the tensioning element upwards in the height direction of the contoured body, and the other receiver, preferably starting from the imaginary centre line of each tensioning element in the longitudinal direction, extends in the direction of the outer edge of the contoured body of the tensioning element downwards in the height direction of the contoured body, wherein each annular receiver has a toothing system on its contact surface pointing towards the imaginary centre line of each tensioning element in the longitudinal direction, wherein individual tensioning elements can be connected to one another such that the receivers, which extend, preferably starting from the imaginary centre line of each tensioning element in the longitudinal direction, in the direction of the outer edge of the contoured body of the tensioning element upwards or downwards in the height direction of the contoured body come to lie one on top of the other such that the contact surfaces of the receivers pointing towards the imaginary centre line of the tensioning element face one another substantially congruently, and wherein the tensioning elements can be aligned with respect to one another in a predetermined angular position by the mutual engagement of the toothing system formed on the mutually facing contact surfaces of the receivers pointing in each case towards the imaginary centre line of each tensioning element in the longitudinal direction.

By means of the fixing device in accordance with the invention, it is advantageously possible to align even complicated fractures, e.g. in the region of the skull or jawbone, in order to positively influence the healing process. In this case, by reason of the spring-elastic deformability of the individual tensioning elements, a defined pressure which can be determined by the material of the tensioning elements can be applied to the ends of the fracture, which contributes to the ends of the fracture growing together in an improved manner. Furthermore, since the tensioning elements can be joined together according to requirements to form chains or more complex structures, e.g. mesh-like structures, and can be precisely aligned by the latching function provided by the complementary toothing system on the receivers, it is possible in particular to fix even the most difficult fractures, e.g. comminuted fractures or the like, which was hitherto not possible with conventional fixing devices. The size of the individual tensioning elements is determined depending upon the field of application. Therefore, larger tensioning elements are used e.g. in the field of spinal column surgery than in the field of facial surgery. Similarly, the chains can also turn out to be longer or shorter depending upon the field of application.

The fixing device in accordance with the invention can advantageously also be aligned during the operation itself, so that, by reason of the modular construction of tensioning elements and sleeve elements, the attending surgeon can quickly adjust e.g. the position of the fixing device and/or the alignment of individual tensioning elements with respect to one another, should this be required by reason of the fracture. For example, when “setting” the fracture the surgeon can change the angular position of the tensioning elements such that the ends of the fracture come to lie cleanly against one another. Since the toothing system of the receivers provides the above-described latching function, the aligned tensioning elements remain in the selected angular position and the fixing device can then be screwed to the bone, without the tensioning elements being “displaced”. This results in improved alignment and fixation of the ends of the fracture, which in turn contributes to the fracture healing without the formation of a callus.

Furthermore, the fixing device in accordance with the invention consists of very few different individual parts. Therefore, the fixing device can be produced simply and cost-effectively. Since the tensioning elements used are also designed with mirror-symmetry along their longitudinal axis, it is possible to produce virtually infinitely long chains or the like from a multiplicity of identical components, i.e. tensioning elements. The number of necessary different parts is thus significantly reduced, while at the same time the field of application of compression-producing fixations of bone fractures is extended.

In accordance with one embodiment of the fixing device in accordance with the invention, the sleeve elements are formed in such a manner that their length can be changed, preferably in a spring-elastic manner, in their axial direction. The sleeve elements can be introduced captively into the receivers and the angular position of the tensioning elements with respect to one another can be changed by means of a latching function, which is implemented by the toothing system formed on the mutually facing receivers, until the fastening means are introduced.

The embodiment of the sleeve elements which can be varied in length advantageously renders it possible that even when the tensioning elements are in the assembled state with the sleeve elements inserted into the receivers thereof, the relative position of the tensioning elements, i.e. their angular position, can continue to be changed. As a result, after aligning the ends of the fracture the attending doctor can still adjust the angular position of the tensioning elements to the extent required for treating the fracture in an optimum manner, in order to avoid the callus formation mentioned in the introduction, i.e. the tyloma-like thickening of the ends of the fracture from overgrowing bone tissue, as the fracture is healing. By screwing the screws which are held in the receivers, more specifically the sleeve elements, into the bone, the sleeve elements are initially pressed together in the longitudinal direction, then compressed and finally optionally slightly squeezed, whereby the relative movability of the tensioning elements with respect to one another is prevented by the fixed mutual engagement of the teeth formed on the receivers. The pre-set angular position of the tensioning elements with respect to one another is thus fixed and the fixing device consisting of a plurality of tensioning elements, sleeve elements etc, is then fastened by screwing all of the receivers to the bone by means of suitable screws, in order to assist the healing process in an optimum manner.

Furthermore, by reason of the sleeve elements which can be captively inserted into the receivers, it is advantageously possible that a theatre nurse, in accordance with the attending doctor's instructions, or even the surgeon himself, will only roughly assemble the required number of individual parts and then “mount the parts on the body” just before the operation. On the contrary, it is possible for the attending doctor and/or the hospital performing the treatment to procure prefabricated “systems”, i.e. fixing devices for bone fractures which consist of tensioning elements, sleeve elements and the like which are of different lengths and are joined together to form chains or the like. For example, such prefabricated fixing devices for bone fractures can be designed as a 2-piece, 3-piece, 4-piece or 5-piece assemblage of tensioning elements and sleeve elements etc. which are then selected shortly before the operation depending upon the type of fracture and the bone to be treated and are provided as part of the preparation for the operation.

In accordance with a further embodiment of the fixing device in accordance with the invention, the surface of the receiver directed to the upper side and/or to the lower side of each tensioning element, or the inner wall region thereof directed to the centre point of the receiver, is concave.

The concave configuration of the surface of the receiver directed to the upper side and/or to the lower side of each tensioning element offers the advantage that the screws used for fixing the fixing device for bone fractures can be screwed-in in such a way that the upper side of the screw head with the tool receiver ideally terminates flush with the receiver, so that there are no projecting edges which could adversely affect the tissue lying thereagainst.

In accordance with another embodiment of the fixing device in accordance with the invention, the toothing system formed on one of the contact surfaces of the mutually facing receivers is complementary to the toothing system formed on the other one of the contact surfaces of the mutually facing receivers.

By forming a complementary toothing system on the surfaces of the receivers of the tensioning elements which can each be moved into mutual abutment, it is possible to ensure exact latching and controllability or adjustability of the angular position of the tensioning elements with respect to one another. In particular, if the toothing system of the receivers is not formed in a complementary manner, the situation can occur that the tensioning elements cannot be arranged in a straight line or in the desired or required angular position with respect to one another. For this reason, provision is made in accordance with the invention that the toothing system, which is formed on the contact surfaces, of the receivers, which can each be moved into abutment with one another, is formed in a complementary manner, so that it is possible to adjust the angular position of the tensioning elements with respect to one another in an exact manner.

In accordance with one embodiment of the fixing device in accordance with the invention, the fastening means are cortical screws and the screw head of each cortical screw is configured, on its lower side facing the thread, preferably in a conical or spherical manner, so that the surface of the screw head pointing towards the receiver is complementary to the surface of the receiver facing the screw head in each case.

By using cortical screws which are typically used in the medical field, it is not necessary in principle to have new screws tested, prior to use in the animal or human body, for their suitability in this regard using cost-intensive measures and to have said screws approved by the committees responsible for this. In a similar manner to the concave configuration of the surface of the receiver directed to the upper side and/or to the lower side of each tensioning element, the conical or spherical configuration of the screw head of the cortical screws offers the advantage that the screws used for fixing the fixing device for bone fractures can be screwed-in in such a way that the upper side of the screw head with the tool receiver ideally terminates flush with the receiver, so that there are no projecting edges which could adversely affect the tissue lying thereagainst.

The tensioning elements formed as described above can be connected, in conjunction with the sleeve elements inserted into the receivers formed thereon, to form almost infinitely long chains. However, in order to be able to fasten these chains to the bone in a stable and reliable manner. “without any offsets or wobble” as it were, it is necessary for those sections of the tensioning elements which hold the screws to come to lie with their entire surface on the bone where possible. Moreover, the cortical screw used for fixing purposes can only be screwed a certain depth into the bone.

Furthermore, in accordance with a further embodiment, the fixing device in accordance with the invention can have at least one termination element which is configured in an annular manner and has a contact surface configured with a toothing system, wherein the termination element further has a sleeve which is integrally formed therewith and which can be introduced through one of the receivers of a tensioning element such that the toothing system of the termination element moves into engagement in a complementary manner with the toothing system formed on the receiver and the tensioning element can be fixed to the bone by introducing the fastening means through the sleeve of the termination element received in the receiver of the tensioning element.

By means of this termination element which can easily be moved into engagement with the receiver of a tensioning element at the respective end of the chain, it is possible in an advantageous manner to ensure that the cortical screw is guided sufficiently and is not screwed too deeply into the bone, while at the same time a stable support surface of the “chain end” on the bone can be provided.

In accordance with another further embodiment of the fixing device in accordance with the invention, at least one of the tensioning elements can have, instead of a receiver at an end in the longitudinal direction of the tensioning elements, an annular receiver which serves as a termination element and into which a sleeve element and/or a fastening means for fixing to the bone can be introduced.

This alternative embodiment for the termination element reduces the number of different parts used and in the same manner achieves the advantages which can be achieved above in conjunction with the separately formed termination element.

In accordance with another embodiment of the fixing device in accordance with the invention, at least one of the tensioning elements is formed with multiple limbs, preferably in a Y-shape, in one piece around a central annular receiving element, and each limb end of such a multiple-limbed tensioning element has a receiver integrally adjoining thereto, on whose contact surface pointing towards the imaginary centre line of each tensioning element in the longitudinal direction a toothing system is formed.

By using such a “complex”, multiple-limbed tensioning element with a plurality (e.g. three, four or more) limbs, it is possible to produce complex arrangements of mutually linked tensioning elements. In this case, multiple combinations of the above-described “simple” tensioning elements and “complex” multiple-limbed tensioning elements (e.g. a Y-assemblage consisting of a multiple-limbed tensioning element and a plurality of simple tensioning elements or a star-like assemblage consisting of a plurality of multiple-limbed tensioning elements and a plurality of simple tensioning elements, including mesh-like structures etc.) can be used in a particularly preferred manner. This is particularly advantageous if, by reason of a complicated fracture e.g. in the region of the wrist joint, multiple bracing of the ends of the fracture and/or bones is required to ensure optimum healing of the fracture.

In accordance with one embodiment of the fixing device in accordance with the invention, at least one of the tensioning elements comprises, instead of one, or both, of the receivers, on at least one end in the longitudinal direction of the tensioning elements, an annular receiving disk which is configured as a toothed disk or the like and which, when linking tensioning elements, sleeve elements and/or fastening means, can be inserted between the annular receivers of other tensioning elements, to form a Y-shaped, star-shaped or other combination of tensioning elements, sleeve elements and/or fastening means.

In a similar manner to the above-described, multiple-limbed tensioning element, it is possible by means of the tensioning element having a receiving disk formed as a toothed disk or the like to produce complex arrangements of multi-linked tensioning elements. Furthermore, the configuration of individual tensioning elements with the receiving disk which is formed as a toothed disk or the like and has a toothing system formed in a complementary manner to the toothing system of the receivers otherwise provided on the tensioning elements offers the advantage that by reason of this “toothed disk” the individual tensioning elements can be joined together, not in angular positions predetermined by the multiple-limbed tensioning element, but rather in arbitrarily selected angular positions with respect to one another to form complex shapes. As a result, the field of application of the fixing device in accordance with the invention, in which such tensioning elements are used, is extended still further.

Further embodiments, features and advantages of the invention will become apparent from the description hereinafter of embodiments with reference to the figures. In the drawing:

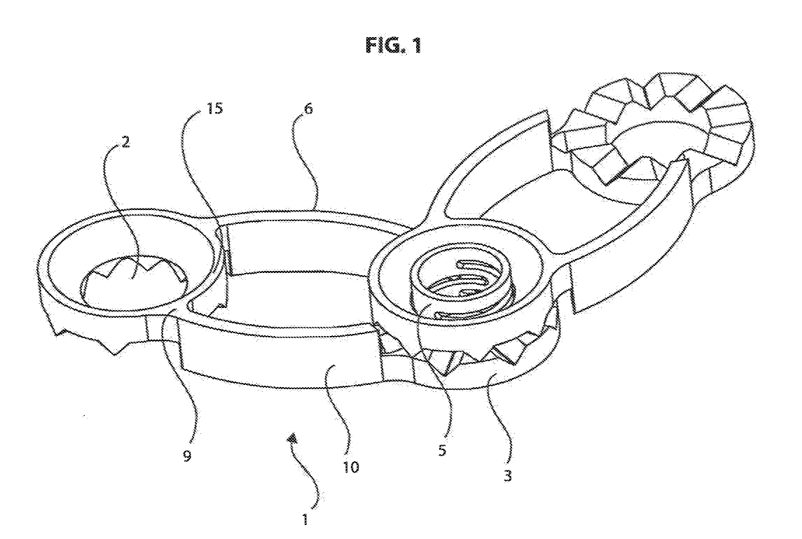

FIG. 1 shows a perspective view of an exemplified embodiment of a fixing device in accordance with the invention disposed loosely;

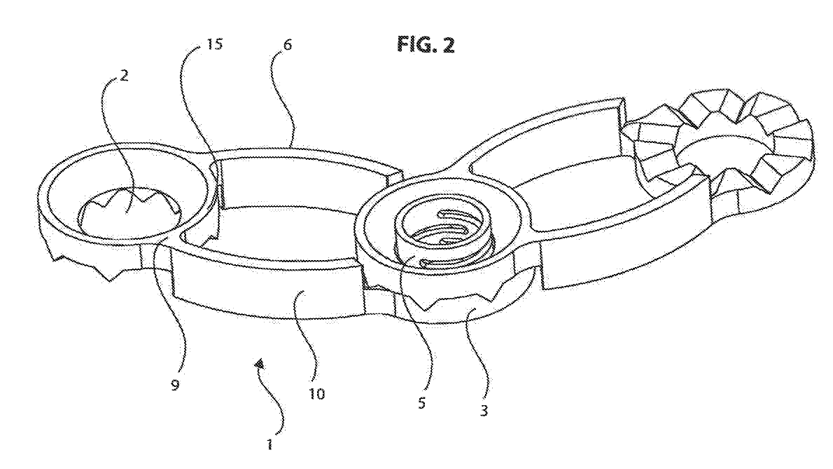

FIG. 2 shows a perspective view of the embodiment of the fixing device of FIG. 1 with intermeshing tensioning elements;

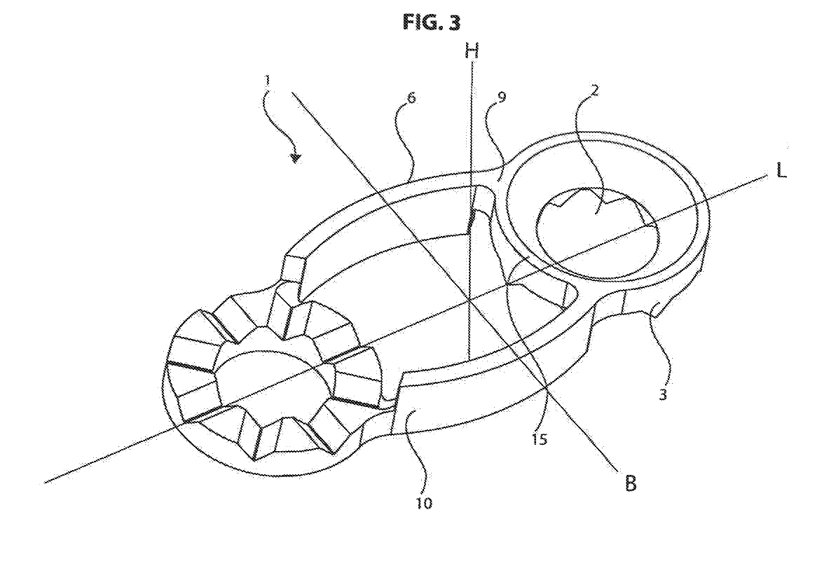

FIG. 3 shows a detailed view of an exemplified embodiment of an exemplified tensioning element which is used in the fixing device in accordance with the invention;

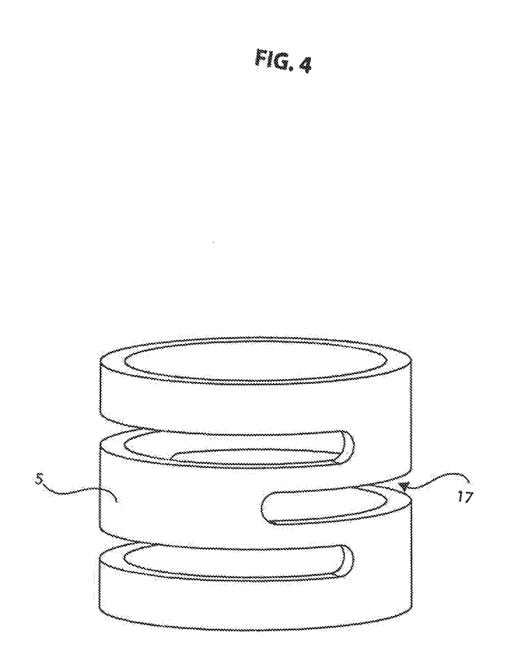

FIG. 4 shows a detailed view of an exemplified embodiment of an exemplified sleeve element which is used in the fixing device in accordance with the invention;

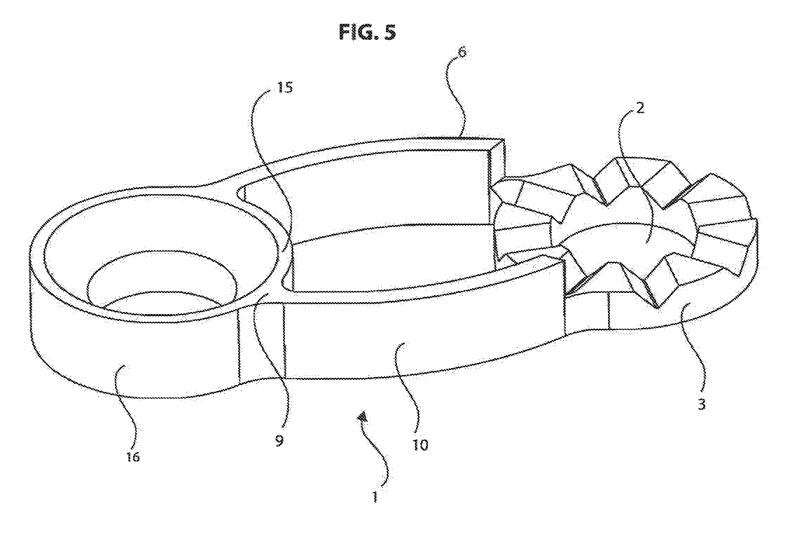

FIG. 5 shows a detailed view of an exemplified embodiment of an exemplified termination element which is used in the fixing device in accordance with the invention;

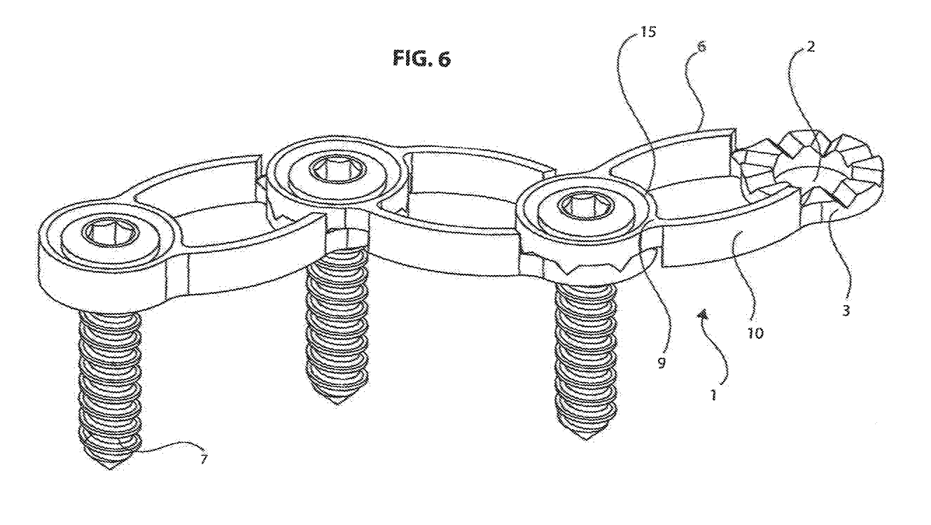

FIG. 6 shows a perspective view of an exemplified embodiment of a fixing device in accordance with the invention comprising a termination element;

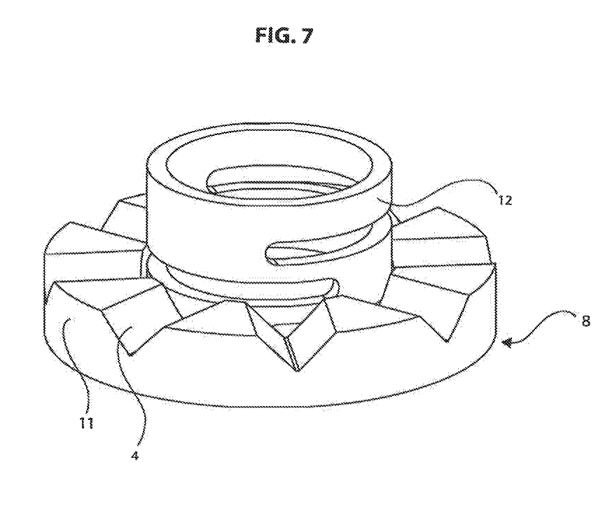

FIG. 7 shows a detailed view of a further exemplified embodiment of an exemplified termination element which is used in the fixing device in accordance with the invention;

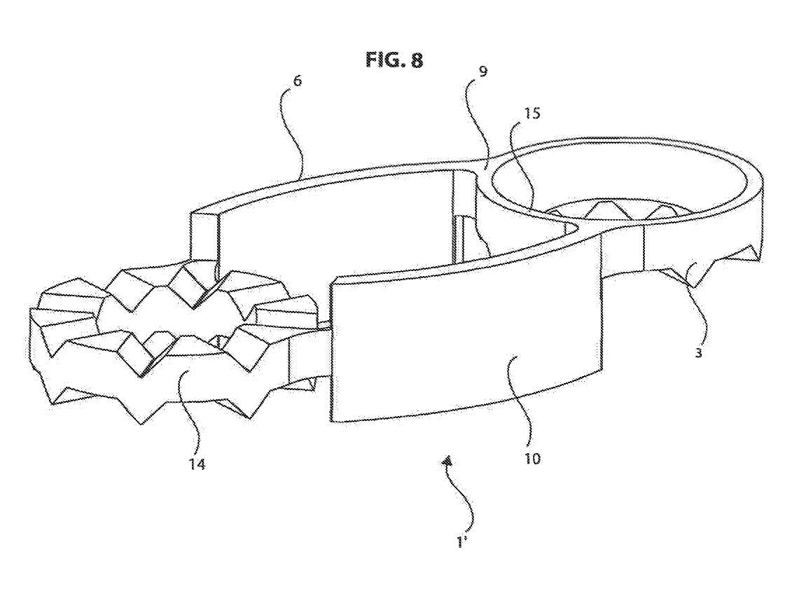

FIG. 8 shows a detailed view of a further exemplified embodiment of an exemplified tensioning element which can be used in the fixing device in accordance with the invention;

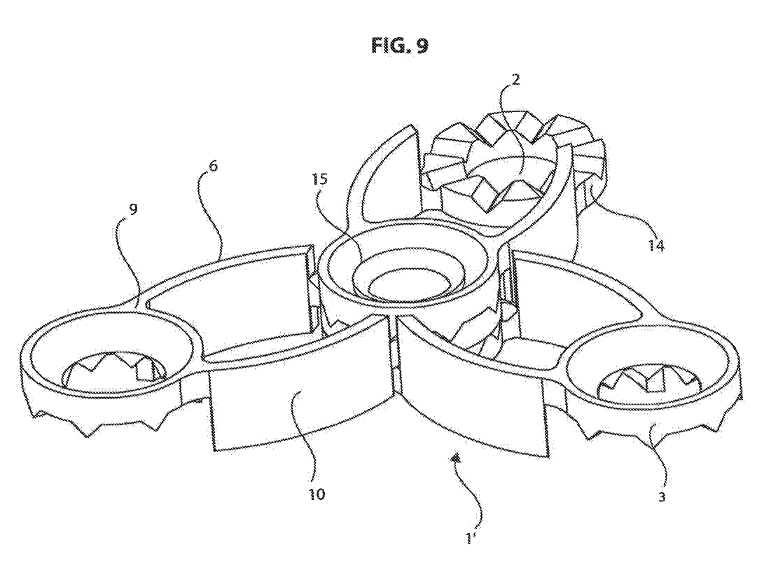

FIG. 9 shows a detailed view of another exemplified embodiment of an exemplified tensioning element which can be used in the fixing device in accordance with the invention; and

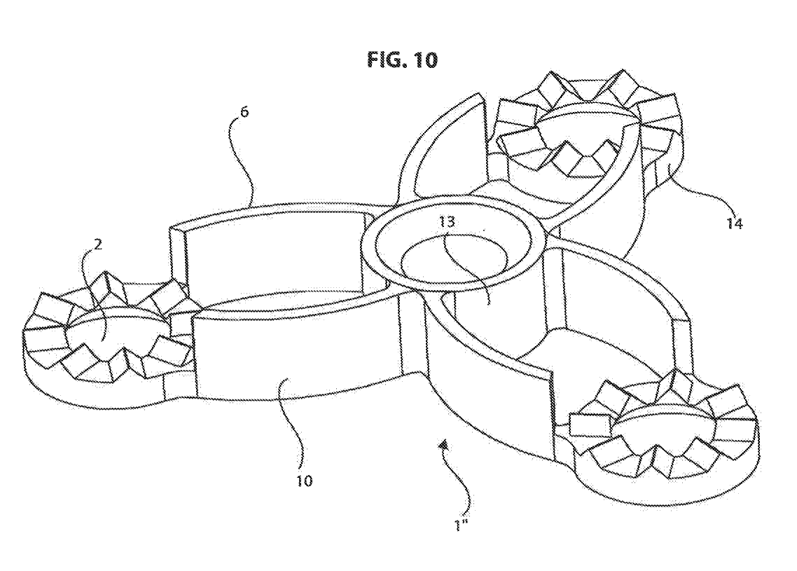

FIG. 10 shows a perspective view of another exemplified embodiment of a fixing device in accordance with the invention;

A preferred embodiment of an exemplified fixing device for bone fractures will be described hereinafter with reference to FIGS. 1 to 3. In this case, FIGS. 1 and 2 show a perspective view of a fixing device for bone fractures which is formed from two tensioning elements 1 which are connected to one another by means of a sleeve element 5. FIG. 3 shows in detail a tensioning element 1 which is used in this embodiment.

It should be noted that in FIG. 1 two tensioning elements 1 are connected to one another “loosely” (i.e. at angles which can be varied) by means of a sleeve element, in order to illustrate the basic principle of the present invention. However, in actual fact the number of tensioning elements 1 required fix the fracture to be treated is determined according to requirements to be e.g. three, four or more tensioning elements 1, and the required tensioning elements 1 are arranged in such a manner that the receivers 2 formed in each case on the tensioning elements 1 overlap one another, as indicated in the figures.

In each case, a sleeve element 5 is introduced through the overlapping receivers 2 and is bent or fixed into place at its upper and lower ends (not illustrated here), so that it is captively field in the overlapping receivers 2.

Each of the receivers 2 of the tensioning element 1 has, on its contact surface pointing towards the imaginary centre line of each tensioning element 1 in the longitudinal direction L, a toothing system 3 by means of which the individual tensioning elements 1 can be aligned with respect to one another in a predetermined angular position.

By virtue of the mutually overlapping arrangement of the receivers 2 of the individual tensioning elements 1, the toothing system 3 comes reciprocally into contact on the contact surfaces directed in each case to the imaginary centre line of the tensioning elements 1 in the longitudinal direction, as shown in FIGS. 1 and 2. The toothing system 3 of the receivers 2 which are each spaced apart from one another in the longitudinal direction of the tensioning element 1 and are arranged inversely with respect to one another is formed in a complementary manner, so that the toothing system 3, pointing in each case towards the centre line, of the mutually spaced apart receivers 2 has the same number of teeth.

Depending upon the desired adjustability of the angles of individual tensioning elements 1 with respect to one another, the toothing system 3 can be formed having more or fewer teeth. This means that the more teeth in the toothing system 3, the more precise the adjustability of the angles of the tensioning elements 1 with respect to one another. In the embodiment illustrated here, the toothing system 3 is formed as a zigzag pattern. However, the toothing system 3 can also be formed as a wave profile, diamond profile or in any other feasible, form-fitting manner, as long as the angular position of the tensioning elements 1 with respect to one another can be correspondingly adjusted and fixed therewith.

FIG. 2 shows the state in which the tensioning elements 1 are aligned at a desired angle and are locked in their position and angular position by the mutual engagement of the toothing system 3 of the individual tensioning elements 1 which is formed on the receivers 2.

The sleeve element 5 which is captively introduced through the mutually overlapping receivers 2 of the individual tensioning elements 1 joins said elements to form a chain in which the individual tensioning elements 1 forms the chain links and are held together by the sleeve element 5 in such a manner as to be rotatable relative to one another about the sleeve element 5.

This means that a sleeve element 5 connects two tensioning elements 1 to one another such that the teeth of the toothing system 3 formed on the receivers 2 do not yet engage one another or do so only slightly and offer a latching function. As a result, the angular position of the tensioning elements 1 with respect to one another can be adjusted, wherein the selected angular position is maintained by reason of the latching function, even if the tensioning elements 1 are no longer held. If a screw 7 (not illustrated here) is screwed through the sleeve element 5 located in the receiver 2, the angular position is fixed, as shown e.g. in FIG. 6, and a further rotation of the tensioning elements 1 relative to one another is not possible.

In order to permit the above-described rotatability of the individual tensioning elements 1 about the sleeve element 5, the sleeve element 5 is formed e.g. in a correspondingly long manner, so that, after being inserted into the receivers 2 and after fixing the ends into place in the longitudinal direction, said sleeve element still provides sufficient “movement clearance” for the mutually connected tensioning elements 1 for a rotation of the tensioning elements 1 and the associated up and down movement along the tooth flanks of the toothing system 3.

However, the sleeve element 5 is formed preferably in a spring-elastic manner in its axial direction, so that the sleeve element (also defined as a spring sleeve) 5 can “stretch or extend” in the longitudinal direction during rotation of the tensioning elements 1 relative to one another for adjustment of the angle, whereby a subsequent adjustment of the angle of the tensioning elements 1 with respect to one another along the graduation of the toothing system 3 on the receivers 2 is possible. An example of such a spring-elastic sleeve element 5 is illustrated in FIG. 4.

In the embodiment illustrated therein, the sleeve element 5 is formed substantially as a circular sleeve whose wall is alternately interrupted by circular segment-like cut-outs 17, in order to provide the spring-elastic properties. As an alternative to the embodiment illustrated here, the sleeve element 5 can also be configured in a similar manner to a coil spring.

As soon as a screw is screwed through the sleeve element 5, in order to fasten the mutually aligned tensioning elements 1 to the bone for the purpose of fixing the bone fracture, the sleeve element 5 is initially pressed together and then compressed and depending upon the screwing-in force is also slightly squeezed under certain circumstances. By screwing the screw into the bone through the spring sleeve held in the receiver 2 and the resulting mutual engagement of the teeth of the toothing system 3 of the receivers 2 formed on the individual tensioning elements 1, the angle of the tensioning elements 1 relative to one another is invariably fixed.

The individual tensioning elements 1 can be arranged into a chain, in which the tensioning elements 1 which are each connected by the sleeve element 5 form the chain links, as part of the preparation for the operation by appropriately trained theatre nurses as specified by the attending doctor.

However, as an alternative it is also feasible for the attending doctor or the hospital performing the treatment to procure prefabricated “chains” in a sterile package suitable for medical operations, wherein these chains are prefabricated by combinations of two, three, four or more tensioning elements 1 with sleeve elements 5 inserted accordingly into the receivers 2 such that the sleeve elements 5, by virtue of their ends being fixed into place in the longitudinal direction, are already captively held in the mutually overlapping receivers 2 but nevertheless permit an adjustment of the angular position of the tensioning elements 1 with respect to one another, as explained above. Ideally, it is then also possible in this case to simultaneously procure the screws which are tailored accordingly to the respective “tensioning element chain” (e.g. cortical screws which are frequently used in the medical field, as well as other screws which are produced specifically for the modular bone plate system, but also nails or dowel systems).

FIG. 3 shows in detail a tensioning element 1 which is used in the fixing device in accordance with the invention.

As shown in FIG. 3, the tensioning element 1 is oval or ellipsoidal and has a contoured body 6 which is hollow in plan view. This contoured body 6 has a circumferential wall with two lateral flanks 10 which lie opposite one another in the width direction B of the tensioning element 1 and which, as seen in plan view, are directed in an arcuate manner outwards from an imaginary centre point of the tensioning element 1. The lateral flanks 10 merge seamlessly into Y-shaped connection points 9 which run out into integrally formed receiving sections 15 which, as seen in plan view, are directed in an arcuate manner inwards to the imaginary centre point of the tensioning element 1.

As can be seen in FIGS. 1 to 3, the individual tensioning elements 1 are formed with mirror-symmetry with respect to their longitudinal direction L, and the mutually spaced apart receivers 2 which lie opposite one another preferably in the longitudinal direction L of the tensioning element 1 are configured such that a receiver 2 of the embodiment of the tensioning element 1 shown here extends, preferably starting from an imaginary centre line of each tensioning element 1 in the longitudinal direction L, in the direction of the outer edge of the contoured body 6 of the tensioning element 1 upwards in the height direction H of the contoured body 6, and the other receiver 2 extends, preferably starting from the imaginary centre line of each tensioning element 1 in the longitudinal direction L, in the direction of the outer edge of the contoured body 6 of the tensioning element 1 downwards in the height direction H of the contoured body 6.

Furthermore, the tensioning element 1 has two mutually spaced apart annular receivers 2 which lie opposite one another preferably in the longitudinal direction L of the tensioning element 1 and which serve to receive the sleeve elements 5 and/or the fastening means 7 (not illustrated here) which can each be inserted through the receivers 2. The receiving sections 15 are an integral component of the annular receivers 2, so that they are configured in one piece with the contoured body 6 and enclose a sleeve element 5 and/or fastening means 7.

In the illustrated embodiment of the tensioning element 1 illustrated in FIG. 3, the receivers 2 are formed on their upper or lower side in a flush manner with the lateral flanks 10 of the tensioning element 1.

Nevertheless, it is not necessary for the receivers to extend from the imaginary centre line of the tensioning elements 1 in the longitudinal direction to the upper and/or lower edge of the contoured body 6. As long as the stability of the receiver 2 can be ensured, the receiver 2 can also be formed in such a manner that its height does not extend completely to the upper and/or lower edge of the contoured body 6 and e.g. a step is formed at the transition between the receiver 2 and the lateral flanks 10, or a flowing transition takes place from the receiver 2 to the lateral flanks 10. Therefore, it is e.g. also feasible that the surface of the receiver directed to the upper or lower side of the contoured body 6 rises at a predetermined angle from its edge lying on the outside in the longitudinal direction of the tensioning element 1 to the lateral flanks 10.

By reason of its use in the medical field, the tensioning element 1 is produced from biocompatible materials. Moreover, a hybrid construction is likewise possible, in which the wall of the contoured body 6 consists in the region of the lateral flanks 10 of a material having the desired elastic property and further regions of the tensioning element 1, such as e.g. the receivers 2, are formed from a rigid or absorbable material. Such a hybrid construction can be achieved e.g. by selecting a corresponding composite profile which, as seen over the cross-section, is composed of sections of different materials. The elastic formation of the lateral flanks 10 makes it possible to introduce a defined tension into each tensioning element 1, thus enabling a compression-producing fixation of bone fractures.

The starting material which can be used for the tensioning element 1 include e.g. metals from the following group: X42CrMo15, X100CrMo17, X2CrNiMnMoNNb21-16-5-3, X20Cr13, X15Cr13, X30Cr13, X46Cr13, X17CrNi16-2, X14CrMoS17, X30CrMoN15-1, X65CrMo 17-3, X55CrMo14, X90CrMoV18, X50CrMoV15, X 38CrMo V15, G-X 20CrMo13, X39CrMo17-1, X40CrMoVN16-2, X105CrMo17, X20CrNiMoS13-1, X5CrNi18-0, X8CrNiS18-9, X2CrNi19-11, X2CrNi18-9, X10CrNi18-8, X5CrNiMo17-12-2, X2CrNiMo17-12-2, X2CrNiMoN25-7-4, X2CrNiMoN17-13-3, X2CrNiMo17-12-3, X2CrNiMo18-14-3, X2CrNiMo18-15-3; X 2 CrNiMo 18 14 3, X13CrMnMoN18-14-3, X2CrNiMoN22136, X2CrNiMnMoNbN21-9-4-3, X4CrNiMriMo21-9-4, X105CrCoMo18-2, X6CrNiTi18-10, X5CrNiCuNb16-4, X3CrNiCuTiNb12-9, X3CrNiCuTiNb12-9, X7CrNiAl17-7, CoCr2ONi15Mo, G-CoCr29Mo, CoCr20W15Ni, Co-20Cr-15W-10Ni, CoCr28MoNi, CoNi35Cr20Mo10, Ti1, Ti2, Ti3, Ti4, Ti-5Al-2,5Fe, Ti-5Al-2,5Sn, Ti-6Al-4V, Ti-6Al-4V EL1, Ti-3Al-2,5V (Gr9), 99,5Ti, Ti-12Mo-6Zr-2Fe, Ti-13,4Al-29Nb, Ti-13Nb-13Zr, Ti-15Al, Ti-15Mo, Ti-15Mo-5Zr-3Al, Ti-15Sn, Ti-15Zr-4Nb, Ti-15Zr-4Nb-4Ta, Ti-15Zr-4Nb-4Ta-0,2Pd, Ti-29Nb-13Ta-4,6Zr, Ti-30Nb-10Ta-5Zr, Ti-35,5Nb-1,5Ta-7,1Zr, Ti-35Zr-10Nb, Ti-45Nb, Ti-30Nb, Ti-30Ta, Ti-6Mn, Ti-5Zr-3Sn-5Mo-15Nb, Ti-3Al-8V-6Cr-4Zr-4Mo, Ti-6Al-2Nb-1Ta-0,8Mo, Ti-6Al-4Fe, Ti-6Al-4Nb, Ti-6Al-6Nb-1Ta, Ti-6Al-7Nb, Ti-6Al-4Zr-2Sn-2Mo, Ti-8,4Al-15,4Nb, Ti-8Al-7Nb, Ti-8Al-1Mo-1V, Ti-11Mo-6Zr-4Sn.

It is also possible to use polymers from the following group: MBS, PMMI, MABS, CA, CTA, CAB, CAP, COC, PCT, PCTA, PCTG, EVA, EVAL, PTFE, ePTFE, PCTFE, PVDF, PVF, ETFE, ECTFE, FEP, PFA, LCP, PMMA, PMP, PHEMA, Polyamide 66, Polyamide 6, Polyamide 11, Polyamide 2, PAEK, PEEK, PB, PC, PPC, PETP, PBT, MDPE, LDPE, HDPE, UHMWPE, LLDPE, PI, PAI, PEI, PIB, POM, PPO, PPE, PPS, PP, PS, PSU, PESU, PVC, PVC-P, PVC-U, ABS, SAN, TPE-U, TPE-A, TPE-E, PVDC, PVA, SI, PDMS, EPM, EP, UF, MF, PF, PUR, UP, PEBA, PHB, PLA, PLLA, PDLA, PDLLA, PGL, PGLA, PGLLA, PGDLLA, PGL-co-poly TMC, PGL-co-PCL, PDS, PVAL, PCL, Poly-TMC, PUR (linear), NiTi Superelastic, NiTi Shape Memory.

It is also possible to use ceramics from the following group: Al₂O₃ (aluminium oxide), Y-TZP (zirconium oxide ceramic), AMC (alumina matrix composite), HA (hydroxilapatite), TCP (tricalciumphosphate), Ceravital (glass ceramic/Bioglas®), FZM/K (zirconium oxide, partially stabilised), TZP-A (zirconium oxide ceramic), ATZ (alumina-toughened zirconia), C799 (aluminium oxide ceramic), Schott 8625 (transponder glass).

It is also possible to use combinations thereof.

Essentially, there are no fixed specifications with regard to the size of the individual tensioning elements 1 and the bone in question. On the contrary, the size of the tensioning elements 1 is determined by the field of application, and thus differs considerably between the application for fixing e.g. bone fractures and facial or skull fractures or the like. Proportional dimensioning of the tensioning element 1 with respect to the screw system used is conceivable as an approximate indicator for the selection of size. From practical medical technology a correlation can be established in approximate terms between the nominal diameter of the cortical or spongiosa screw and the field of application, which is listed in the following table.

| Screw system | Fields of application |
|---|---|
| HA 1.5 Cortical | Oral and maxillofacial surgery |
| HA 2.0 Cortical | Cranio; foot surgery; hand surgery |
| HA 2.5 Cortical | Cranio; foot surgery; trauma |
| HA 3.5 Cortical | Thorax; lower back; arm surgery |
| HA 4.0 Cortical | Thorax; spinal column; hip and pelvis area |
| HA 4.5 Cortical | Leg surgery; trunk; fibula; shoulder |
| HA 5.0 Cortical | Leg surgery; tibia; femur |
| HB 4.0 Spongiosa | depending on the load case |
| HB 6.0 Spongiosa | depending on the load case |

The selection of the dimensioning and positioning of the screw system and of the tensioning element 1 also depends upon the type of fracture (e.g. transverse fracture, oblique fracture) and the location of the fracture, which results in different load cases.

Thus, in case of diaphyseal fractures, in most cases cortical systems will be used, as here no spongiosa proportion is given. On the other hand, in case of fractures near a joint very often spongiosa systems will be used, as the percentage of the spongiosa is very high in this region. Spongiosa screws have a higher percentage contact area, as they have a larger core diameter for the same nominal diameter. In the case of joint fractures, i.e. fractures which include the articular surface, cortical systems as well as spongiosa systems will be used in dependence upon the local anatomic conditions. In case of a multiple fracture, both systems can also be used.

Moreover, apart from a single, i.e. monocortical fixation possibility, also bicortical systems can be provided in which the screw is fixed through the bone at both cortical regions.

As shown in FIG. 6, tensioning elements 1 which serve as termination elements are provided on the ends of a tensioning element chain and allow the ends of the fixing device thus assembled to be fixed to the bone with corresponding screws 7. FIG. 5 shows an exemplified embodiment of such a tensioning element 1 with a termination element. As can be seen in FIG. 5, this termination element is constructed as far as possible in the manner of a conventional tensioning element 1 but has, at one end in the longitudinal direction, an annular receiver 16 into which the sleeve element 5 and a screw 7 for fixing to the bone can be introduced, instead of the receiver 2 which has the toothing system 3. As also in the case of the receivers 2 provided with the toothing system, at least the contact surface facing the screw head, or the inner wall region directed to the receiver centre point, of the annular receiver 17 is concave, so that the screw head can be screwed-in as flush as possible with respect to the upper edge of the receiver 2 (of the annular receiver 17) and thus avoids any irritation of surrounding tissue. Since, in the case of the termination elements, the individual tensioning elements 1 do not have to be able to rotate with respect to one another, it is also possible to introduce the screw 7 without an interposed sleeve into the annular receiver 17 and then to screw the tensioning element 1 to the bone.

An alternative embodiment of a termination element 8 is illustrated in FIG. 7. This termination element 8 is configured in an annular manner and has a contact surface 11 configured with a toothing system 4 and has a sleeve 12 which is provided in one piece therewith and can be introduced through one of the receivers 2 of a tensioning element 1, so that the toothing system 4 of the termination element 8 moves into engagement in a complementary manner with the toothing system 3 formed on the receiver 2 and the tensioning element 1 can be fixed to the bone by introducing a screw 7 through the sleeve 12 of the termination element 8 received in the receiver 2 of the tensioning element 1.

Such termination elements are necessary for ensuring that the ends of the “chain”, which is assembled from different tensioning elements 1 and sleeve elements 5 lie smoothly on the bone.

Instead of or in addition to the tensioning elements 1 described above in particular in conjunction with FIGS. 1 to 3, it is also possible to use a tensioning element 1′, which is shown in FIG. 8, for forming the fixing device in accordance with the invention.

This tensioning element 1′ differs from the above-described tensioning element 1 in that, instead of one, or both, of the receivers 2, an annular receiving disk 14 which is configured as a toothed disk or the like is formed on at least one end in the longitudinal direction of the tensioning element 1′ and when linking tensioning elements, sleeve elements 5 and/or screws 7, can be inserted between the annular receivers 2 of other tensioning elements 1, as shown schematically in FIG. 9. By linking one or a plurality of tensioning elements 1, 1′, it is possible to produce in a simple manner e.g. Y-shaped or star-shaped tensioning elements, sleeve elements and/or screws, or another combination thereof.

A further embodiment of a tensioning element is illustrated in FIG. 10. This tensioning element 1″ differs from the tensioning elements explained above in that it has a multiple-limbed shape instead of the oval shape. In particular, the multiple-limbed tensioning element 1″ illustrated in FIG. 10 is integrally formed in a Y-shape about a central annular receiving element 13 and each limb end of the multiple-limbed tensioning element 1″ has a receiver 2 integrally adjoining thereto, on whose contact surface pointing towards the imaginary centre line of the tensioning element 1″ in the longitudinal direction L the toothing system 3 is formed, by means of which, as explained above, a latching function is produced which permits alignment of the tensioning elements with respect to one another and at the same time, as soon as the tensioning elements are screwed, ensures that the desired angular position is fixed.

Although not illustrated in the figures, the multiple-limbed tensioning element 1″ can also be formed with four, five or more limbs. The tensioning elements 1, 1′ and 1″ described herein can be combined with one another in an arbitrary manner, so that in addition to the chain or star arrangements discussed above, every feasible arrangement from an arbitrary combination of the individual tensioning elements 1, 1′ and 1″ can be provided. Therefore, it is also possible to produce complex “networks” from a combination of all three of the tensioning elements 1, 1′ and 1″ illustrated here, wherein either the separate termination elements 8 shown in FIG. 7 or the tensioning elements shown in FIG. 6 with the integrated annular receiver 16 serving as a termination element are used on the respective ends of the linked tensioning elements 1, 1′ and 1″.

The present invention provides, as illustrated above, a fixing device for bone fractures having at least two tensioning elements 1, 1′ and 1″, at least two fastening means 7 and at least two sleeve elements 5. Each of the tensioning elements 1, 1′ and 1″ has a contoured body 6 and has, at ends mutually spaced apart in the longitudinal direction, receivers 2 which enclose sleeve elements 5 and/or fastening elements 7, so that by linking a plurality of tensioning elements 1, sleeve elements 5 and/or fastening means 7 they can be arranged into a chain or the like. Each tensioning element 1 is formed with point-symmetry in the width direction or with mirror-symmetry with respect to the longitudinal direction, wherein the receivers 2 extend, starting from an imaginary centre line of each tensioning element 1 in the longitudinal direction L in each case upwards or downwards to the outer edge of the contoured body 6 of the tensioning element 1 in the height direction H and have a toothing system 3 on their contact surface facing the imaginary centre line, wherein, when connecting individual tensioning elements 1 to one another, said elements can be aligned with respect to one another in a predetermined angular position by the mutual engagement of the toothing system 3 formed on the mutually facing contact surfaces of the receivers 2.
1, 1′, 1″ . . . tensioning element
2 . . . annular receiver
3, 4 . . . toothing system
5 . . . sleeve element
6 . . . contoured body
7 . . . fastening means
8 . . . termination element
9 . . . connection point
10 . . . lateral flank
11 . . . contact surface
12 . . . sleeve
13 . . . receiving element
14 . . . annular receiving disk
15 . . . receiving section
16 . . . annular receiver
17 . . . cut-out
B . . . width direction
L . . . longitudinal direction
H . . . height direction

1.-12. (canceled)

13. A fixing device for bone fractures, wherein the device comprises:
at least two tensioning elements, at least two fastening elements which can be introduced into the bone, and at last two sleeve elements, wherein
each tensioning element comprises:
a contoured body which is hollow in plan view, has a circumferential wall and has two lateral flanks which lie opposite one another in width direction (B) of the tensioning element and which, seen in plan view, are directed in an arcuate manner outwards from an imaginary center point of the tensioning element,
wherein the two lateral flanks merge seamlessly into Y-shaped connection points which run out into integrally formed receiving sections which, seen in plan view, are directed in an arcuate manner inwards to the imaginary center point of the tensioning element,
at least two mutually spaced apart annular receivers which lie opposite one another and serve to receive the sleeve elements and/or the fastening elements which can each be inserted through the receivers,
wherein the receiving sections are an integral component of the annular receivers, so that they are configured in one piece with the contoured body and enclose a sleeve element and/or fastening element, and wherein
by linking a plurality of tensioning elements, sleeve elements and/or fastening elements they can be arranged into a chain;
wherein the circumferential wall of the contoured body of each tensioning element is deformable in a spring-elastic manner at least in sections in a region of the lateral flanks,
wherein each tensioning element is formed with mirror-symmetry with respect to a longitudinal direction (L) of the tensioning element and the mutually spaced apart receivers which lie opposite one another are configured in such a manner that
one receiver extends in a direction of an outer edge of the contoured body of the tensioning element upwards in a height direction (H) of the contoured body, and
the other receiver extends in the direction of the outer edge of the contoured body of the tensioning element downwards in the height direction (H) of the contoured body,
wherein each annular receiver comprises a toothing system on its contact surface pointing towards the imaginary center line of each tensioning element in longitudinal direction (L),
wherein individual tensioning elements can be connected to one another such that the receivers, which extend in a direction of the outer edge of the contoured body of the tensioning element upwards or downwards in a height direction (H) of the contoured body come to lie one on top of the other such that contact surfaces of the receivers pointing towards the imaginary center line of the tensioning element face one another substantially congruently,
and wherein the tensioning elements can be aligned with respect to one another in a predetermined angular position by mutual engagement of the toothing system formed on mutually facing contact surfaces of the receivers pointing in each case towards the imaginary center line of each tensioning element in longitudinal direction (L).

14. The device of claim 13, wherein the at least two fastening elements can be introduced into the bone in a region of ends of a fracture.

15. The device of claim 13, wherein the at least two mutually spaced apart annular receivers lie opposite one another in longitudinal direction (L) of the tensioning element.

16. The device of claim 13, wherein the receivers, starting from an imaginary center line of each tensioning element in longitudinal direction (L), extend in a direction of the outer edge of the contoured body of the tensioning element upwards or downwards in a height direction (H) of the contoured body.

17. The device of claim 13, wherein the sleeve elements are formed in such a manner that their length can be changed in their axial direction.

18. The device of claim 17, wherein the sleeve elements can be changed in their axial direction in a spring-elastic manner.

19. The device of claim 13, wherein the sleeve elements can be captively introduced into the receivers.

20. The device of claim 13, wherein an angular position of the tensioning elements with respect to one another can be changed by means of a latching function, which is implemented by the toothing system formed on the mutually facing receivers, until the fastening elements are introduced.

21. The device of claim 13, wherein a surface of the receiver directed to an upper side and/or to a lower side of each tensioning element is concave.

22. The device of claim 13, wherein the toothing system formed on one of the contact surfaces of the mutually facing receivers is complementary to the toothing system formed on the other one of the contact surfaces of the mutually facing receivers.

23. The device of claim 13, wherein the fastening elements are cortical screws.

24. The device of claim 23, wherein a screw head of each cortical screw is configured in a conical or spherical manner, so that a surface of the screw head pointing towards the receiver is complementary to a surface of the receiver facing the screw head in each case.

25. The device of claim 13, wherein the device further comprises at least one termination element which is configured in an annular manner and has a contact surface configured with a toothing system, wherein the termination element further comprises a sleeve which is integrally formed therewith and which can be introduced through one of the receivers of a tensioning element such that the toothing system of the termination element moves into engagement in a complementary manner with the toothing system formed on the receiver and the tensioning element can be fixed to the bone by introducing the fastening element through the sleeve of the termination element received in the receiver of the tensioning element.

26. The device of claim 13, wherein at least one of the tensioning elements comprises, instead of a receiver at an end in longitudinal direction of the tensioning elements, an annular receiver which serves as a termination element and into which a sleeve element and/or a fastening element for fixing to the bone can be introduced.

27. The device of claim 13, wherein at least one of the tensioning elements is formed with multiple limbs in one piece around a central annular receiving element, and each limb end of such a multiple-limbed tensioning element has a receiver integrally adjoining thereto, on whose contact surface pointing towards the imaginary center line of each tensioning element in the longitudinal direction a toothing system is formed.

28. The device of claim 27, wherein at least one of the tensioning elements is formed with multiple limbs in a Y-shape.

29. The device of claim 13, wherein at least one of the tensioning elements comprises, instead of one, or both, of the receivers, on at least one end in longitudinal direction of the tensioning elements, an annular receiving disk which is configured as a toothed disk and which, when linking tensioning elements, sleeve elements and/or fastening elements, can be inserted between the annular receivers of other tensioning elements, to form a combination of tensioning elements, sleeve elements and/or fastening elements.

30. The device of claim 29, wherein the combination of tensioning elements, sleeve elements and/or fastening elements is Y-shaped.

31. The device of claim 29, wherein the combination of tensioning elements, sleeve elements and/or fastening elements is star-shaped.
